(19) United States
(12) Patent Application Publication
(10) Pub. No.: US 2025/0281546 A1
(43) Pub. Date: Sep. 11, 2025

(54) Multi-Nutrient Delivery Composition

(71) Applicant: Nutrient Foods, LLC, Reno, NV (US)

(72) Inventor: Charles H. Marsland, Windermere, FL (US)

(21) Appl. No.: 18/601,016

(22) Filed: Mar. 11, 2024

(51) Int. Cl. A61K 35/60; A23L 19/00; A61K 9/00; A61K 31/07; A61K 31/355; A61K 31/375; A61K 33/00; A61K 33/06; A61K 33/26; A61K 47/46

(52) U.S. Cl. CPC A61K 35/60; A23L 19/09; A61K 9/0056; A61K 31/07; A61K 31/355; A61K 31/375; A61K 33/00; A61K 33/06; A61K 33/26; A61K 47/46

(57) ABSTRACT

A multi-nutrient delivery composition including a base that includes fig and may also include tapioca. Vitamins, minerals and fish oil are encapsulated by the base. The base is configured to act as both a textural agent with compression strength between 5-8 LBF and an encapsulation material for the vitamins, minerals, and fish oil, inhibiting oxidation of the fish oil from both the atmosphere and oxidizing minerals included in the multi-nutrient composition.

### BACKGROUND

This disclosure relates to a multi-nutrient delivery composition that has both desirable measured long-term compression strength and tested organoleptic olfactory properties.

Multi-nutrient delivery is an ever-growing and more modern method of consumption for today's on-the-go consumers as well as for those with pill and capsule fatigue. The use of formats including ready-to drink liquids, nutrient bars, powdered drink mixes, baked goods, and confections including chocolate and gummies have been available since the advent of fortification. However, these formats deliver nutrients mostly limited to vitamins, minerals and proteins. The same compositional formats have been modified to include cannabidiol (CBD) and tetrahydrocannabinol (THC). All of these compositions, regardless the compositional format, have been unable to deliver vitamins, minerals, protein and fish oil-based omega 3 fatty acids together in one delivery format with long shelf life and non-fish odor smell, without gelatin encapsulation of the fish oil, in the formulation until now.

### SUMMARY

Aspects and examples are directed to the use of a novel combination of materials and methods of making an improved multi-nutrient delivery composition that has both desirable measured long-term compression strength and tested organoleptic olfactory properties. This is important because multi-nutrient delivery systems are an ever-growing and more modern method of consumption for today's on-the-go consumer as well as for those with pill and capsule fatigue. The use of formats including ready-to-drink liquids, nutrient bars, powdered drink mixes, baked goods, and confections including chocolate and gummies have been available since the advent of fortification. However, these formats deliver nutrients mostly limited to vitamins, minerals and proteins. The same compositional formats have been modified to include CBD and THC. All of these compositions, regardless of the compositional format, have been unable to deliver vitamins, minerals, protein and Omega 3 Fatty Acids together in one delivery format with long shelf life and non-fish odor and or smell, without gelatin encapsulation of the fish oil, until now. The uniqueness of our multi-nutrient delivery composition is that the need for expensive gelatin encapsulated fish-based omega-3 oil is not required, the products have consistent compression strength through its lifecycle of 1.5 years after manufacturing, and there is no unacceptable or below satisfactory olfactory odors from the fish based omega-3 oil that is not encapsulated and directly folded into the composition matrix.

The multi-nutrient delivery composition is based on the use of fig, the edible fruit of Ficus Carica, a species of small tree in the flowering plant family Moraceae, native to the Mediterranean region, together with western and southern Asia and cultivated since ancient times and is now widely grown throughout the world, in combination with high purity fish-based omega-3 oil. The unique combination was found to be more effective than the use of gelatin encapsulated fish-based omega-3 in a regular multi-nutrient food system including nutrient bars, baked goods, confections, or gummies. The unique combination has been paired successfully with vitamins, minerals, including and plant-based omega-3 oil materials making the multi-nutrient delivery composition superior to all other formats.

The uniqueness of the composition is due the consumer-friendly organoleptic qualities including taste and appearance but those are trumped by the composition's desirable smell and texture extending through its entire shelf life in unmodified conditions and environments meaning no refrigeration. The levels of omega-3 from fish are also to be noted as very high.

The novel use of fig is viewed as a direct replacement for the materials used in other multi-nutrient delivery compositions including chewable supplements which have been manufactured and sold in the form a gummy candy and/or nutrition bar. Today, there is wide use of this format for vitamins, minerals and dietary supplements. Traditional gummy manufacturing is complicated and not simple. It requires the use of a gelatin base which serves as the bridging agent that gives the candy its desired compression strength. In addition to the gelatin, gummy compositions are also made using water, a sugar source (e.g., corn syrup, and/or sugar), flavors, and colors. When mass produced, a gelatin base or stock is first mixed and pumped into a special candy cooker that cooks the gelatin base by steam. Then, the cooker pumps the gelatin base into a vacuum chamber to remove excess moisture. From the vacuum chamber, the cooked candy moves to a mixing station where colors, flavors, acids, and fruit concentrates are mixed into the cooked candy. Next, a starch molding machine, commonly known as a mogul, pumps the candy stock into starch filled mold boards that shape the candies. After curing, the gummies are removed from the molds and then packaged, delivered, and sold. During manufacturing, the gummy candy may be cooked to temperatures exceeding 200° F. These high temperatures will heat sensitive ingredients, especially fish-based omega 3 oil, to oxidize and smell like fish. This eliminates this option as acceptable.

The second form of multi-nutrient delivery in food form is a nutrition bar a/k/a protein bar a/k/a energy bar. This format, unlike gummies, is made either through sheeting, extrusion, rotary molding or molding. All these methodologies share similar ingredient base compositions including a base material (e.g. protein powder, nut, dairy powder, marshmallow, grain, puffed grain, puffed protein crisp, cookie, wafer, cereal piece, dry fruit, root flour, tuber flour, starch, resistant starch, legume, and or fruit paste), a binder (e.g. fruit juice syrup, fruit paste, gum base, sugar syrup, fructose syrup, allulose syrup, soluble fiber, tapioca syrup, molten sugar, sugar alcohol syrup, caramel, chocolate, polydextrose, tagatose, grain syrup, nut paste, and or fat), inclusions (e.g. confections, fruit pieces, nuts, pastes, puffed materials, crisp materials, chocolate materials, grains, cookies, gummies, seeds, fibers, and flavor inclusion pieces), additional layers (caramel, cookie, grain, coatings, nut paste, fruit paste, sugar coating) and an optional exterior fat coating (chocolate, compound coating, panned sugar coating, and or shellac). These delivery forms are made including vitamins, some minerals and may include omega 3 from plants but rarely fish oil omega 3 unless encapsulated.

The uniqueness of the use of fig as both the encapsulation and long-term compression strength agent in a nutrition bar, confection, gummy like piece, as well as inclusion for other food items and bars is novel.

The invention entails the novel use of fig, the edible fruit of Ficus Carica, a species of small tree in the flowering plant family Moraceae, in a multi-nutrient composition to maintain desirable consistent compression strength for at least one year and up to two years while simultaneously encapsulating fish oil, vitamins and minerals maintaining satisfactory organoleptic properties, as evaluated through a Taste Panel Evaluation Methodology, that rates appearance, taste/flavor, odor/aroma, and texture. The lack of odor and fish smell according to panel participants validates the unique application of fig as an encapsulation material for fish oil but also vitamins and oxidizing minerals. This novel application of fig as a textural, compression and encapsulating agent for multi-nutrients has not been utilized before in combination.

### DETAILED DESCRIPTION

The invention entails the novel use of fig, the edible fruit of Ficus Carica, a species of small tree in the flowering plant family Moraceae, in a multi-nutrient composition to maintain desirable consistent compression strength for up to 2 years while simultaneously encapsulating fish oil, vitamins and minerals maintaining satisfactory organoleptic properties, as evaluated through a Taste Panel Evaluation Methodology, that rates appearance, taste/flavor, odor/aroma, and texture. The lack of odor and fish smell according to panel participants validates the unique application of fig as an encapsulation material for fish oil but also vitamins and oxidizing minerals. This novel application of fig as a textural, compression and encapsulating agent for multi-nutrients has not been utilized before in combination.

The compression strength of the compositions are detailed at temperature.

| Sample | | Taste | Compression Strength Test 2 | | |
|---|---|---|---|---|---|
| (C is for chocolate | | panel | (the (months) is the number of | | |
| bar and PB is for | Compression | Odor | months that test 2 was run after | | Taste panel |
| peanut butter bar) | Strength Test 1 | rating | test ! had been run) | | Odor rating |
| — | LBF/TEMP (F.) | — | LBF/TEMP (F.) | | — |
| 1-10092023 (C) | 8.1/77 | S | 8.97/77 | (14 months) | S |
| 2-8272025 (C) | 6.95/77 | S | 7.00/77 | (2 months) | S |
| 3-7192024 (C) | 7.25/76.6 | S | 7.45/76.8 | (10 months) | S |
| 4-10092023 (C) | 6.85/77 | S | 7.05/76.9 | (2 months) | S |
| 5-7192024 (PB) | 7.14/76.8 | S | 7.40/77 | (10 months) | S |
| 6-6152024 (PB) | 6.10/77.1 | S | 6.35/77 | (10 months) | S |
| 7-7052024 (C) | 7.41/77.2 | S | 7.61/77 | (10 months) | S |
| 8-7052024 (C) | 8.11/76.8 | S | 8.46/77 | (12 months) | S |
| 9-7192024 (PB) | 7.25/76.8 | S | 7.83/77 | 12 months ( ) | S |

The importance of compression strength corresponds to the organoleptic qualities of the multi-nutrient delivery composition. The compression force, which is better described texturally as “the bite”, needs to be consistent over time to meet desirability characteristics. If the composition hardens, in this case going up 25+% the characteristics are negative. Remaining consistent is the objective. To test this, the compression strength is measured with corresponding temperature of the composition at time of measurement. All tests for these compositions were done in a controlled atmosphere to guarantee consistency. In addition to the compression strength measurements, the composition was also evaluated for appearance, smell, and odor. The specific focus was on fish aroma and smell. This is an acceptable practice used in food testing and the methods followed were those established as an industry standard and are done so by a scale of 1. Unacceptable, 2. Below Satisfactory, 3. Satisfactory, 4. Above Satisfactory, 5. Good. On all tests, the compositions tested 3. Satisfactory with no fish odor. Below is an Organoleptic Taste Panel Form used to evaluate the product.

Following are two specific non-limiting examples of the multi-nutrient delivery compositions.

### Example 1: Fig with Peanut Bar

### Ingredient: Amount (Grams)

40 lb. Natural Seedless Fig Paste—Nutra Fig 14.0046
Tapioca Syrup Organic DE 60 12.0042
De-Hulled Hemp Seed 5.0016
Simply Natural 1M Peanut Drop—11849 5.0016
Semi-Sweet Chocolate Chips 4.0014
ARCON T Soy Flake Concentrate 3.670674
Oats, Regular Rolled Gluten Free #5 2.348251
Cold Milled 30 Mesh Golden Flaxseed 2.0004
Organic Brown Rice Crisped 1.977132
Partially Defatted Peanut Flour—28% Fat (Light Roast) 1.5006
Bovine Collagen Hydrolysate 1.5006
VEGETABLE GLYCERIN USP 99.7% 1.423205
EPAX—4030 TGN Fish Oil 1.0968
Kleptose Betadex Cyclodextrin—Item #341001 1.0002
Water 0.948804
Non-GMO Expeller Pressed RBD Canola Oil 0.593065
Cold Milled 30 Mesh Golden Flaxseed 0.474402
Mineral Calcium Lactate Pentahydrate; 98-101% Assay; 5743-47-5 CAS 0.313494
Mineral Potassium Gluconate; 99-101% Assay; 299-27-4
CAS 0.237195
Natural Peanut Butter Flavor Oil Soluble: OC-05078 0.204
Mineral Dipotassium Phosphate; min. 98% Assay 0.120695
Mineral Tripotassium Citrate; 99-100.5% Assay; 6100-05-6 CAS 0.109484
Nat. Masking Flavor, Oil Soluble 0.0852
Vitamin Choline Bitartrate Coated; 98˜99.5% Assay; 87-67-2 CAS 0.083391
Mineral Microencapsulated Magnesium Oxide 40%; 36.8-44% Assay; 19.2-26.7% Mg 0.061419
Purified Sea Salt Powder 0.04739
Vitamin C (ascorbic acid) 0.045904
Natural Vanilla Type Flavor Powder (Non-GMO) 0.033261
Vitamin E 50% (dl-alpha-tocopheryl acetate) 0.024295
Palm Kernel Oil 0.014555
Vitacel Oat Fiber HF200 0.011589
Biotin (1% on maltodextrin) 0.010914
Vitamin A (retinyl palmitate) 0.008937
Niacin (niacinamide) 0.007691
Maltodextrin (IP) 0.006131
Pantothenic Acid (calcium-D-pantothenate) 0.005213
Vitamin K1 (1% on mannitol) 0.004993
Vitamin D3 (cholecalciferol) 0.003333
Mineral Ferrochel® (Ferrous Bisglycinate Chelate); 20% Fe 0.003001
Mineral Zinc Citrate Dihydrate; min. 31.3% Assay; 5990-32-9 CAS 0.002551
Folic Acid (10% on maltodextrin IP) 0.001782
Mineral Copper Bisglycinate Chelate; 10% CU 0.001161
Vitamin B1 (thiamine mononitrate) 0.000942
Vitamin B6 (pyridoxine hydrochloride) 0.000939
Mineral Selenium L-Methionine Complex 5,000 MCG/G
HFG; 0.5% Se 0.000881
Vitamin B2 (riboflavin) 0.000761
Mineral Manganese Sulfate 1 H2O UPS/FCC Powder; 98˜102% Assay; 10034-96-5 CAS 0.000381
Vitamin B12 (cyanocobalamin 1% on maltodextrin IP) 0.000249
Mineral Molybdenum Trituration 5%; 5-6.3% Mo 9.32E-05
Mineral Chromium Chloride Hexahydrate, Milled; NLT 96% 4.24E-05
Mineral Potassium Iodide UPS; 99-101.5% Assay 8.47E-06

### Example 2: Fig and Chocolate Bar

Ingredient Amount (grams)
40 lb. Natural Seedless Fig Paste-Nutra Fig 13.999206
Tapioca Syrup Organic DE 60 12.499356
De-Hulled Hemp Seed 4.9995
Semi-Sweet Chocolate Chips 4.49955
ARCON T Soy Flake Concentrate 3.669310947
Infused Dried Cranberry Bits 3.49965
Cocoa Powder Processed with Alkali 2.69973
Oats, Regular Rolled Gluten Free #5 2.347379057
Cold Milled 30 Mesh Golden Flaxseed 1.9998
Organic Brown Rice Crisped 1.976398171
Bovine Collagen Hydrolysate 1.49985
VEGETABLE GLYCERIN USP 99.7% 1.4226767
EPAX—4030 TGN Fish Oil 1.096254
Water 0.948451133
Non-GMO Expeller Pressed RBD Canola Oil 0.592844455
Cold Milled 30 Mesh Golden Flaxseed 0.474225567
Mineral Calcium Lactate Pentahydrate; 98-101% Assay; 5743-47-5 CAS 0.313377748
Mineral Potassium Gluconate; 99-101% Assay; 299-27-4 CAS 0.237106542
Natural Lemon Flavor WONF, Oil Soluble 0.127866
Mineral Dipotassium Phosphate; min. 98% Assay 0.120649798
Mineral Tripotassium Citrate; 99-100.5% Assay; 6100-05-6 CAS 0.109442911
Nat. Masking Flavor, Oil Soluble 0.085446
Vitamin Choline Bitartrate Coated; 98˜99.5% Assay; 87-67-2 CAS 0.083360245
Mineral Microencapsulated Magnesium Oxide 40%; 36.8-44% Assay; 19.2-26.7% Mg 0.061396457
Purified Sea Salt Powder 0.047372559
Vitamin C (ascorbic acid) 0.04579814
Natural Vanilla Type Flavor Powder (Non-GMO) 0.033248287
Vitamin E 50% (dl-alpha-tocopheryl acetate) 0.024238909
Palm Kernel Oil 0.01454961
Vitacel Oat Fiber HF200 0.011584503
Biotin (1% on maltodextrin) 0.010889239
Vitamin A (retinyl palmitate) 0.008916021
Niacin (niacinamide) 0.007673625
Maltodextrin (IP) 0.006116975
Pantothenic Acid (calcium-D-pantothenate) 0.005201012
Vitamin K1 (1% on mannitol) 0.004981766
Vitamin D3 (cholecalciferol) 0.003325237
Mineral Ferrochel® (Ferrous Bisglycinate Chelate); 20% Fe 0.002999845
Mineral Zinc Citrate Dihydrate; min. 31.3% Assay; 5990-32-9 CAS 0.002549715
Folic Acid (10% on maltodextrin IP) 0.001778332
Mineral Copper Bisglycinate Chelate; 10% CU 0.001160501
Vitamin B1 (thiamine mononitrate) 0.000940324
Vitamin B6 (pyridoxine hydrochloride) 0.000936669
Mineral Selenium L-Methionine Complex 5,000 MCG/G HFG; 0.5% Se 0.000880965
Vitamin B2 (riboflavin) 0.000758836
Mineral Manganese Sulfate 1 H2O UPS/FCC Powder; 98˜102% Assay; 10034-96-5 CAS 0.000381187
Vitamin B12 (cyanocobalamin 1% on maltodextrin IP) 0.000248479
Mineral Molybdenum Trituration 5%; 5-6.3% Mo 9.31789E-05
Mineral Chromium Chloride Hexahydrate, Milled; NLT 96% 4.23541E-05
Mineral Potassium Iodide UPS; 99-101.5% Assay 8.47081E-06

These exemplary bars are made as follows. The base material is a mixture of the fig and the tapioca. However, the base material could consist only of the fig paste. The rest of the ingredients are folded into the base material utilizing a process referred to as folding, also known as lamination sheeting. This encapsulates the other ingredients in the base. It is then rolled out and cut into bars. Lamination sheeting folds the fig and tapioca base, engulfing the fish oil and vitamins and minerals and encapsulating the fish oil sufficiently that the odor of the oil is undetectable for at least one year.

In some examples, the mass balance ratio of fig to fish oil is 14:1; this results in complete, organoleptically acceptable encapsulation of the fish oil.

In another example an additional 1 g of omega-3 from plant-based sources is included in the ingredients that are folded into the base material. In this example the 14:1 fig:fish oil ratio becomes 14:1+1; this still results in a total encapsulation of the fish plus plant source simultaneously, equaling a compression strength stable long shelf life bar.

Examples of the compositions, systems, methods and apparatuses discussed herein are not limited in application to the details of construction and the arrangement of components set forth in the following description or illustrated in the accompanying drawings. The compositions, systems, methods and apparatuses are capable of implementation in other examples and of being practiced or of being carried out in various ways. Examples of specific implementations are provided herein for illustrative purposes only and are not intended to be limiting. In particular, functions, components, elements, and features discussed in connection with any one or more examples are not intended to be excluded from a similar role in any other examples.

Examples disclosed herein may be combined with other examples in any manner consistent with at least one of the principles disclosed herein, and references to “an example,” “some examples,” “an alternate example,” “various examples,” “one example” or the like are not necessarily mutually exclusive and are intended to indicate that a particular feature, structure, or characteristic described may be included in at least one example. The appearances of such terms herein are not necessarily all referring to the same example.

Also, the phraseology and terminology used herein is for the purpose of description and should not be regarded as limiting. Any references to examples, components, elements, acts, or functions of the compositions, systems and methods herein referred to in the singular may also embrace embodiments including a plurality, and any references in plural to any example, component, element, act, or function herein may also embrace examples including only a singularity. Accordingly, references in the singular or plural form are not intended to limit the presently disclosed systems or methods, their components, acts, or elements. The use herein of “including,” “comprising,” “having,” “containing,” “involving,” and variations thereof is meant to encompass the items listed thereafter and equivalents thereof as well as additional items. References to “or” may be construed as inclusive so that any terms described using “or” may indicate any of a single, more than one, and all of the described terms.

Having described above several aspects of at least one example, it is to be appreciated that various alterations, modifications, and improvements will readily occur to those skilled in the art. Such alterations, modifications, and improvements are intended to be part of this disclosure and are intended to be within the scope of the invention. Accordingly, the foregoing description and drawings are by way of example only, and the scope of the invention should be determined from proper construction of the appended claims, and their equivalents.

What is claimed is:

1. A multi-nutrient delivery composition comprising: fig, vitamins, minerals and fish oil, wherein the fig is configured to act as both a textural agent with compression strength between 5-8 LBF and an encapsulation material for the vitamins, minerals, and fish oil, inhibiting oxidation of the fish oil from both the atmosphere and oxidizing minerals included in the multi-nutrient composition.

2. The multi-nutrient composition of claim 1 comprising a high-purity omega-3 fish oil.

3. The multi-nutrient composition of claim 2 comprising a bar with up to 2.0 grams of the high-purity omega-3 fish oil.

4. The multi-nutrient composition of claim 1 that meets satisfactory standards of organoleptic testing for odor and smell for up to one year after manufacturing.

5. The multi-nutrient delivery composition of claim 1 made by lamination sheeting followed by reduction rolling, forming and cutting to a unit with a defined size and weight thereby delivering a specific quantity of fish oil per unit.

6. The multi-nutrient composition of claim 1 wherein the fish oil percentage is 1.66% of the total weight of the composition.

7. The multi-nutrient delivery composition of claim 1 comprising vitamins A, C, E, and minerals calcium, potassium, and iron.

8. The multi-nutrient delivery composition of claim 1 comprising a base made from fig and tapioca, wherein the rest of the ingredients are folded into the base by the process of lamination sheeting.

9. The multi-nutrient delivery composition of claim 8 wherein the lamination sheeting process encapsulates the other ingredients in the base.

10. A method of making a food bar, comprising:
developing a base material comprising fig paste;
adding to the base material a plurality of additional ingredients comprising fish oil, vitamins, and minerals;
folding the additional ingredients into the base material by lamination sheeting to develop an intermediate material; and
rolling the intermediate material out to a desired thickness and then cutting it into bars.

11. The method of claim 10, wherein the base material further comprises tapioca.

12. The method of claim 11, wherein the additional ingredients further comprise a source of plant-based omega 3 fatty acid.
